(19) United States
(12) Patent Application Publication
(10) Pub. No.: US 2026/0294775 A1
(43) Pub. Date: Oct. 1, 2026

(54) PRE-TREATMENT COMPOSITION FOR PROTECTING HAIR DURING OXIDATIVE COLORING

(71) Applicant: Daniel Villano, New York, NY (US)

(72) Inventor: Daniel Villano, New York, NY (US)

(73) Assignee: Daniel Villano, New York, NY (US)

(21) Appl. No.: 19/218,454

(22) Filed: May 26, 2025

Related U.S. Application Data
(60) Provisional application No. 63/779,457, filed on Mar. 28, 2025.

(51) Int. Cl. A61K 8/66; A61K 8/46; A61K 8/49; A61Q 5/00

(52) U.S. Cl. CPC A61K 8/66; A61K 8/46; A61K 8/4986; A61Q 5/004

(57) ABSTRACT

The present disclosure provides a pre-treatment composition for protecting hair during oxidative coloring. The composition comprises a transglutaminase enzyme, a thiol donor selected from N-acetyl cysteine ethyl ester (NAC-EE), DL-homocysteine thiolactone (HCTL), and/or (R)-2-oxothiazolidine-4-carboxylic acid (OTC); and a cosmetically acceptable carrier. The pre-treatment composition may further include one or more of a penetration enhancer and a conditioning agent. Upon application to hair before oxidative coloring, the transglutaminase enzyme catalyzes the formation of internal isopeptide bonds within keratin fibers, reinforcing hair structure. During the oxidative coloring process, the thiol donor releases thiol groups under alkaline conditions to repair disulfide bonds in real time, thereby minimizing chemical damage. A method for protecting hair is also disclosed, comprising applying the pre-treatment composition to hair, leaving it in place, and subsequently subjecting the hair to oxidative coloring without rinsing off the composition.

### CROSS-REFERENCE TO RELATED APPLICATIONS

This application claims the benefit of U.S. Provisional Patent Application No. 63/779,457, filed on Mar. 28, 2025, and titled “Multi-Phase System for Protecting and Repairing Hair During Oxidative Coloring” which is incorporated herein by reference in its entirety.

### FIELD OF THE INVENTION

The present disclosure relates to the field of hair care compositions and, more particularly, to a pre-treatment composition configured to protect and reinforce hair structure prior to exposure to oxidative coloring agents, thereby minimizing damage during chemical treatments.

### BACKGROUND OF THE DISCLOSURE

Hair coloring has been an essential part of personal grooming and beauty for centuries, evolving over time to incorporate more sophisticated techniques and chemical formulations. Hair coloring, particularly oxidative hair coloring, is a widely used technique for altering hair color in both professional salons and at-home treatments. The process involves the use of oxidizing agents such as hydrogen peroxide and persulfates, which penetrate the hair shaft to remove or modify natural pigments and deposit artificial color. However, this process induces significant damage to the hair structure, leading to weakened, brittle, and porous hair fibers.

One of the primary structural damages caused by oxidative coloring is the breakage of disulfide bonds within the keratin matrix of the hair. Disulfide bonds are essential for maintaining the hair's strength, elasticity, and resilience. When exposed to oxidative agents, these bonds break, leading to a loss of protein integrity, resulting in weak and fragile hair. The reduction in disulfide bonds increases brittleness, making hair more prone to breakage and split ends. Additionally, as the elasticity of the hair decreases, it loses its natural strength and flexibility, leading to an overall compromised structure.

Another critical issue associated with oxidative coloring is cuticle damage. The hair cuticle serves as the outermost protective layer, shielding the inner cortex from environmental stressors. During oxidative treatments, high-pH chemicals lift the cuticle scales to allow color penetration. However, this also leads to increased porosity, causing the hair to absorb and lose moisture more rapidly. The disruption of the cuticle structure weakens the lipid barrier, resulting in frizz, roughness, and dullness. Furthermore, once the cuticle is compromised, the hair becomes highly susceptible to mechanical and chemical stress, accelerating long-term damage and making it difficult to maintain smooth and healthy strands.

Current hair protection and repair solutions attempt to address these concerns but present significant limitations. Single-step bond-building treatments aim to reconnect broken disulfide bonds but fail to provide continuous protection throughout the oxidative process. Post-color protein treatments attempt to rebuild the keratin structure after damage has already occurred, offering no real-time bond reinforcement. Lipid-restoring masks and conditioners primarily focus on surface-level hydration but do not address internal protein degradation. These existing solutions fail to provide a comprehensive approach that protects hair before the oxidative coloring process.

Numerous patents have explored various methods of bond repair during oxidative coloring, but none of them offer a solution that prevents structural damage before chemical exposure. For example, U.S. Pat. No. 3,533,417 discusses the use of homocysteine thiolactone in permanent waving, but it is applied in a reducing environment aimed at reshaping hair, rather than preserving disulfide bonds under oxidative conditions. U.S. Pat. No. 5,490,980 (Unilever) reveals the use of transglutaminase in cosmetic compositions, but this approach does not combine the enzyme with thiol-based bond builders in the context of bleaching or dyeing, missing the opportunity for a continuous protective effect. Olaplex™ patents (e.g., U.S. Pat. No. 9,498,419) employ maleic acid derivatives in bleach to reconnect disulfide bonds but do not incorporate enzymatic crosslinking nor provide sustained protection for the cuticle and lipid layers. Similarly, K18 Peptide (WO 2018/149267) focuses on repairing polypeptide chains post-damage but lacks continuous protection during the oxidative process, leaving the hair still vulnerable to oxidative stress and damage.

Additionally, oxidative stress during coloring leads to free radical generation, which further weakens hair proteins and accelerates long-term fragility. Free radicals cause irreversible damage to the hair structure, and conventional treatments do not include antioxidant mechanisms to mitigate oxidative damage in real time. As a result, hair that undergoes frequent oxidative coloring remains vulnerable to cumulative degradation, leading to progressive weakening over time.

Hence, there is a need for an improved composition that addresses the structural damage caused by oxidative hair coloring, including the breakage of disulfide bonds, cuticle disruption, and cumulative oxidative stress, while providing better protection for hair integrity throughout the coloring process.

### BRIEF SUMMARY OF THE DISCLOSURE

The present disclosure provides a pre-treatment composition for protecting hair during oxidative coloring. The pre-treatment composition is formulated to reinforce the keratin structure of hair prior to exposure to oxidative agents and to activate during the oxidative coloring process to repair disulfide bonds, thereby minimizing structural damage and preserving hair strength and elasticity.

In an embodiment, the pre-treatment composition comprises a transglutaminase enzyme that catalyzes the formation of isopeptide bonds within the keratin matrix of hair fibers prior to chemical treatment. The composition further includes a thiol donor selected from the group consisting of N-acetyl cysteine ethyl ester (NAC-EE), DL-homocysteine thiolactone (HCTL), (R)-2-oxothiazolidine-4-carboxylic acid (OTC), and combinations thereof, which remain latent until exposure to oxidative conditions, at which point thiol groups are released to facilitate the repair of broken disulfide bonds. A cosmetically acceptable carrier is used to deliver the active components effectively into the hair structure.

In some embodiments, the pre-treatment composition may further comprise one or more additional components, including penetration enhancers to facilitate deep delivery of active agents into the hair cortex and conditioning agents to improve the manageability and cosmetic feel of the hair. Suitable penetration enhancers include dimethyl isosorbide (DMI), and suitable conditioning agents include betaine and arginine methyl ester. The pre-treatment composition may be formulated as a spray, lotion, serum, or cream, and is maintained within a pH range of about 6.0 to about 7.0 to optimize transglutaminase activity without premature activation of thiol donors.

The present disclosure further provides a method for protecting hair during oxidative coloring. The method comprises applying the pre-treatment composition to hair for a period of time sufficient to allow penetration into the hair fiber, typically ranging from about two hours to overnight, and subsequently subjecting the hair to an oxidative coloring treatment without removing the pre-treatment composition. During the oxidative coloring process, the elevated pH and presence of oxidizing agents activate the thiol donors, resulting in real-time repair of disulfide bonds concurrent with oxidative exposure.

The pre-treatment approach disclosed herein pre-emptively strengthens the hair structure before chemical insult, mitigates oxidative and mechanical damage during coloring, and preserves the hair's tensile strength, elasticity, and cuticle integrity. This system represents a significant advancement over conventional post-damage repair treatments by addressing the root cause of oxidative weakening in a preventive manner.

### DETAILED DESCRIPTION

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, firmware, and/or by human operators.

### Terminology

Brief definitions of terms used throughout this application are given below.

The terms “connected” or “coupled” or “attached”, and related terms are used in an operational sense and are not necessarily limited to a direct connection or coupling. Thus, for example, two devices/equipments/components may be coupled directly, or via one or more intermediary devices/equipments/components. As another example, devices/equipments/components may be coupled in such a way that information can be passed there between, while not sharing any physical connection with one another. Based on the disclosure provided herein, one of ordinary skill in the art will appreciate a variety of ways in which connection or coupling exists in accordance with the aforementioned definition.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

Exemplary embodiments will now be described more fully hereinafter with reference to the accompanying drawings, in which exemplary embodiments are shown. This disclosure may, however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

The present disclosure provides a multi-phase system designed to protect and repair hair during oxidative coloring treatments. The system may be formulated to address hair damage at multiple stages through one or more compositions, including a pre-treatment composition, a bond-repair composition, and post-treatment composition. Each phase incorporates specific compositions to maintain the integrity of the hair structure during chemical treatments involving oxidative agents such as bleaching and dyeing. In some embodiments not claimed herein, additional compositions for bond repair, antioxidant protection, or post-color restoration may be employed.

In an embodiment, the pre-treatment composition is applied to the hair before the oxidative coloring process to enhance the structural integrity of keratin, reinforce hair bonds, and provide an internal protective network against chemical damage. In an embodiment, the pre-treatment composition comprises a transglutaminase enzyme, a thiol donor selected from the group consisting of N-acetyl cysteine ethyl ester (NAC-EE), DL-homocysteine thiolactone (HCTL), (R)-2-oxothiazolidine-4-carboxylic acid (OTC), and combinations thereof, and a cosmetically acceptable carrier. In some embodiments, the composition may further comprise one or more of a penetration enhancer and a conditioning agent. The pre-treatment composition is configured for pre-application, wherein the composition is applied to the hair and left on for a period ranging from several hours to overnight before oxidative coloring. The composition does not interfere with the coloring process and ensures that the hair is primed for the subsequent oxidative treatment.

In an embodiment, the pre-treatment composition comprises a transglutaminase enzyme. The transglutaminase enzyme catalyzes the formation of isopeptide bonds between glutamine and lysine residues within keratin proteins, reinforcing the internal protein structure of the hair before exposure to oxidative chemicals. The transglutaminase enzyme may be present in an amount ranging from 0.3% to 2.0% by weight of the total composition. The enzyme operates effectively in a pH range of 6 to 7, ensuring optimal protein crosslinking without inducing unwanted structural modifications to the hair. Upon application, transglutaminase pre-loads the hair with internal crosslinks, effectively fortifying the keratin fibers against future oxidative damage. In a further embodiment, the pre-treatment composition may optionally contain keratin peptides or protein fragments to enhance the crosslinking reaction. These peptides provide additional binding sites for transglutaminase activity, further reinforcing the protein scaffold inside the hair fiber. The inclusion of these peptides ensures that even chemically weakened hair benefits from enzymatic crosslinking.

In an embodiment, the pre-treatment composition includes a thiol donor selected from the group consisting of N-acetyl cysteine ethyl ester (NAC-EE), DL-homocysteine thiolactone (HCTL), (R)-2-oxothiazolidine-4-carboxylic acid (OTC), and combinations thereof as thiol donors. These compounds pre-load the hair fiber with thiol precursors, which become activated during the oxidative process to mitigate the effects of disulfide bond breakage.

### N-Acetyl Cysteine Ethyl Ester (NAC-EE)

NAC-EE is a lipophilic, esterified cysteine derivative that is more permeable to the hair fiber compared to standard cysteine-based treatments. NAC-EE remains largely inert upon application but undergoes hydrolysis under oxidative alkaline conditions (such as exposure to bleach or oxidative dye formulations), releasing N-acetyl cysteine (NAC) inside the hair. Chemical Mechanism of NAC-EE Activation: Under high-pH conditions (pH 8.5 or above), NAC-EE hydrolyses to produce N-acetyl cysteine, which acts as a free thiol donor. The thiol groups (—SH) released by NAC can participate in thiol-disulfide exchange reactions, reconnecting broken disulfide bonds (—S—S—) and stabilizing the protein structure of the hair. This process provides continuous bond reinforcement during the oxidative stage. The NAC-EE content in the pre-treatment composition ranges from 1.0% to 3.0% by weight, ensuring sufficient thiol reservoirs within the hair cortex.

In an additional embodiment, after the oxidative treatment is rinsed out, a near-neutral enzymatic cross-link mask may be applied to the hair to finalize internal repairs as a reinforcement step. The reinforcement step may operate at around pH 6-7 to allow transglutaminase (TGA) continued activity in bridging keratin proteins:
1. Transglutaminase (additional dose) at ~0.2-2.0% by weight,
2. Protein substrates (e.g., hydrolyzed keratin 1-2%, peptides) for TGA to graft onto hair,
3. Minimal cationic or humectant components to maintain manageability but not inhibit enzymatic function.

In an embodiment, the mechanism for reinforcement step may include catalyzing TGA isopeptide bond formation between lysine and glutamine residues over a short contact time (such as 5-10 minutes). This reinforcement step may address residual microdamage, creating new covalent cross-links that fortify the hair's internal matrix beyond the disulfide bond level. By the end of phase 3, hair has both newly formed disulfide/thiol-based bonds from the coloring phase and enzyme-driven isopeptide bonds, leading to a multi-tier bonding network.

In an embodiment, the method of application for the reinforcement step includes lightly shampooing (if desired), after rinsing out the color/bleach, then applying the near-neutral TGA mask. The applied near-neutral TGA mask is then allowed to sit for 5-10 minutes under ambient or mild heat conditions. Thereafter, the hair may be rinsed thoroughly. This reinforcement steps may effectively “patch” any final weak points prior to the acidic sealing. It may be noted that the TGAS does not do well in acidic conditions, thus this reinforcement steps is for further bonding, if needed, but it cannot be in the final acidic conditioner because it will not work. Therefore, this would be where high lift bleach (many levels) or multiple bleach sessions

### DL-Homocysteine Thiolactone (HCTL)

In a further embodiment, the pre-treatment composition comprises DL-Homocysteine Thiolactone (HCTL), a cyclic precursor of homocysteine. HCTL acts as a dual-action bond builder, wherein it remains largely inert upon initial application but undergoes ring-opening under alkaline oxidative conditions, releasing homocysteine.

### Dual Mechanism of HCTL Activation:

(a) Disulfide Exchange Mechanism: Homocysteine released from DL-homocysteine thiolactone behaves similarly to cysteine by facilitating thiol-disulfide exchange reactions that result in the re-formation of broken disulfide bonds within the keratin matrix. Under alkaline oxidative conditions encountered during coloring, the free thiol (—SH) group of homocysteine reacts with oxidized cysteine residues to restore the structural disulfide bridges (—S—S—) critical for hair strength and elasticity.
(b) N-Homocysteinylation: Unlike NAC, homocysteine possesses an additional amine (—NH₂) functional group, allowing it to form amide bonds with keratin proteins. This process, termed N-homocysteinylation, involves the covalent attachment of homocysteine to lysine residues within keratin chains. The formation of these permanent amide bonds leads to enhanced structural reinforcement of the hair fiber, providing both immediate and long-term protection against mechanical and chemical damage.

In an embodiment, the concentration of DL-homocysteine thiolactone (HCTL) in the pre-treatment composition is maintained at 0.5% to 1.0% by weight, ensuring an adequate thiol reservoir for oxidative stress protection. The controlled release of homocysteine during the oxidative coloring process provides continuous disulfide bond restoration, thereby minimizing structural damage to hair fibers and preserving hair integrity throughout the chemical treatment.

In an embodiment, the pre-treatment composition may further comprise penetration enhancers to facilitate deep fiber penetration of active ingredients into the hair cortex. Suitable penetration enhancers include Dimethyl Isosorbide (DMI), which improves the solubility and diffusion of active compounds within the hair shaft. In an embodiment, the DMI is included at a concentration ranging from about 1.0% to about 2.0% by weight of the composition. The use of penetration enhancers ensures that thiol donors and enzymatic crosslinkers reach sufficient depth in the keratin structure to perform their protective functions effectively during the oxidative process.

In a further embodiment, the pre-treatment composition may include mild conditioning agents to enhance the hair's manageability and provide additional cosmetic benefits without interfering with the protective mechanism. Suitable conditioning agents include:

Betaine (~1.0%)—Provides scalp comfort and moisture retention.

Arginine Methyl Ester (~1.0%)—Enhances ionic bonding between protein structures, reducing static and frizz.

General ionic bonding compound, Fumaric acid or fumarate derivatives, Acrylamido based diacids with L-arg-ome (~1.0%)—Functions as a supplementary ionic bonding agent, complementing NAC-EE and HCTL activity.

In an exemplary embodiment, the pre-treatment composition comprises the following formulation:

| Ingredient | Concentration (% w/w) |
|---|---|
| Transglutaminase | 0.3-2.0% |
| N-acetyl cysteine ethyl ester (NAC-EE) | 1.0-3.0% |
| DL-homocysteine thiolactone (HCTL | 0.5-1.0% |
| Dimethyl Isosorbide (DMI) | 1.0-2.0% |
| An ionic bonding stabilizer | ~1.0% |
| Betaine | ~1.0% |
| Arginine Methyl Ester | ~1.0% |
| Water & pH Adjusters | qs to 100 |

### Method of Application:

In an embodiment, the method of application for the pre-treatment composition involves applying the composition to dry or damp hair several hours to overnight before the oxidative coloring procedure. The composition is not rinsed out prior to coloring, ensuring that the enzymatic crosslinks, pre-loaded thiols, and penetration enhancers remain in the hair structure, actively protecting against oxidative damage. The pre-treatment composition pre-loads the hair with a protective network, reinforcing keratin before oxidative exposure, ensuring real-time repair during coloring, and minimizing post-color damage. By integrating enzymatic crosslinking, time-release thiol mechanisms, and deep penetration enhancers, this composition addresses oxidative damage at its root, providing a novel and comprehensive protection system.

In an alternative embodiment, the pre-treatment composition may be provided as two distinct formulations, designed to address different levels of oxidative stress and hair conditions while remaining within the scope of the present disclosure. The first formulation may correspond to a Pre-Base Color Scalp Spray, formulated to protect the scalp and roots during oxidative coloring treatments, particularly for individuals with sensitive scalps or frequent color applications. The second formulation may correspond to a Pre-Highlight Protector, specifically designed for high-damage oxidative processes such as bleaching, high-lift coloring, and highlights, providing enhanced keratin reinforcement prior to exposure to strong oxidizing agents.

### Pre-Highlight Protector:

The Pre-Highlight Protector is a structurally reinforcing formulation, optimized for pre-bleach and pre-highlift treatments, where the risk of disulfide bond breakage and cuticle degradation is highest. This formulation comprises: 1. Bond Reinforcement Agents: Transglutaminase (TGA) (~0.3-2.0%)—Strengthens keratin through enzymatic crosslinking, and N-Acetyl Cysteine Ethyl Ester (NAC-EE) (~1.0-3.0%)—Preloads the hair with thiol donors for oxidation resistance. 2. Protein and Amino Acid Complex: Hydrolyzed Silk (~0.3-0.8%)—Provides additional structural reinforcement, and Arginine (~0.5-1.5%)—Helps maintain keratin integrity under oxidative conditions. 3. Cuticle & Ionic Stabilizers: An ionic bonding stabilizer (~0.5-1.5%)—Enhances ionic stabilization in the hair cortex, and Betaine (~1.0%)—Maintains moisture balance and elasticity. The pH of the Pre-Highlight Protector is maintained between 6.5 and 7.0, making it ideal for pre-bleach application without disrupting developer activity. This formulation is applied on dry or damp hair before bleaching or high-lift coloring and left in to ensure pre-emptive bond reinforcement.

In an embodiment, the bond-repair composition is designed for real-time protection and repair of hair bonds during oxidative coloring. The bond-repair composition is formulated to be mixed directly into the oxidative hair color or bleach to actively repair and reinforce the keratin structure as disulfide bonds break during processing. In an embodiment, the bond-repair composition comprises a disulfide bond-repair agent, an ionic bond stabilizer, a pH buffer system, and optional hydrogen bond reinforcers and antioxidants. The bond-repair composition functions within the alkaline oxidative environment (pH 7-10) of hair color and bleaching formulations, ensuring real-time bond restoration without interfering with color lift or dye penetration.

In a specific embodiment, the bond-repair composition comprises DL-Homocysteine Thiolactone (HCTL) as the primary bond-repair agent. HCTL is a latent thiol donor, meaning it remains inactive in neutral or mildly acidic conditions but activates under alkaline oxidative conditions, such as the high pH environment of bleaching (pH 9-10).

### DL-Homocysteine Thiolactone (HCTL) Mechanism

In an exemplary mechanism, HCTL undergoes ring-opening at high pH, releasing homocysteine, which contains free thiol (—SH) groups. These thiols facilitate thiol-disulfide exchange reactions, actively reconnecting broken disulfide bonds (—S—S—) during oxidative coloring. In a further embodiment, homocysteine provides dual repair functionality:
(a) Disulfide Bond Repair: Homocysteine reacts with oxidized cysteine residues, re-linking disulfide bridges within keratin proteins.
(b) Permanent Protein Grafting: Homocysteine contains an amine (—NH₂) functional group, which can form amide bonds with lysine residues in keratin, permanently anchoring structural reinforcements.

The HCTL concentration in the bond-repair composition is maintained at 0.5% to 3.0% by weight, ensuring optimal thiol availability without interfering with the oxidative process.

### Synergistic Role of N-Acetyl Cysteine Ethyl Ester (NAC-EE)

In another embodiment, the bond-repair composition includes N-Acetyl Cysteine Ethyl Ester (NAC-EE), which serves as a secondary thiol donor to capture and stabilize broken disulfide bonds in real-time. NAC-EE remains stable in neutral conditions but hydrolyses in alkaline oxidative environments, releasing N-acetyl cysteine (NAC), which participates in thiol-disulfide exchange reactions. In a preferred embodiment, the NAC-EE content ranges from 1.0% to 5.0% by weight, ensuring sufficient thiol reserves for continuous bond restoration during the oxidative treatment.

In another embodiment, the bond-repair composition further includes an ionic bonding stabilizer that functions as an ionic crosslinker, which temporarily reinforces protein structures by forming ionic bridges between keratin residues during the oxidative process. In a preferred embodiment, the ionic bonding stabilizer is included at a concentration of 1.0% to 2.5% by weight to stabilize the ionic balance in hair without disrupting the oxidative coloring process. Such ionic bonding stabilizer may, without any limitation, include L-arginine methyl ester and Acrylic Acid-2-Acrylamido-2-Methylpropane Sulfonic Acid (AMPS) copolymer.

In another embodiment, the bond-repair composition includes a pH buffer system to maintain the bleaching or dye mixture at an optimal alkalinity range (pH 9-10). Suitable buffering agents include: Tris(hydroxymethyl)aminomethane (TRIS), and Amino acid buffers (e.g., glycine, arginine derivatives).

Function of the Buffer System: 1. Prevents excessive alkalinity, which can degrade keratin structure. 2. Stabilizes the pH of the bleach/dye mixture, ensuring consistent lift and color penetration. 3. Ensures the optimal activation of HCTL and NAC-EE without destabilizing the oxidative process.

In a preferred embodiment, the buffer system is included at 0.5% to 2.0% by weight, maintaining an alkaline pH suitable for oxidative reactions while minimizing collateral protein damage.

In another embodiment, the bond-repair composition further comprises hydrogen bond reinforcers to strengthen non-covalent interactions within hair keratin. Suitable reinforcers include: 1. Creatine (~0.5% to 2.0%)—Stabilizes ionic and hydrogen bonds in keratin, 2. Polyquaternium polymers (~0.5% to 1.5%)—Provides electrostatic reinforcement to protect protein structures.

In a further embodiment, the bond-repair composition includes antioxidants to mitigate free radical damage caused by oxidative stress. Suitable antioxidants include: 1. Hydroxytyrosol (~0.3-0.5%)—Neutralizes free radicals without interfering with color oxidation, 2. Vitamin E (~0.3%)—Reduces lipid oxidation in the hair cuticle.

In an exemplary embodiment, the bond-repair composition comprises:

| Ingredient | Concentration (% w/w) |
|---|---|
| DL-Homocysteine Thiolactone | 0.5-3.0% |
| N-Acetyl Cysteine Ethyl Ester | 1.0-5.0% |
| An ionic bonding stabilizer | 1.0-2.5% |
| Buffer System (TRIS, Glycine) | 0.5-2.0% |
| Creatine | 0.5-2.0% |
| Polyquaternium Polymer | 0.5-1.5% |
| Antioxidants (Hydroxytyrosol, Vitamin E) | 0.3-0.5% |
| Water & pH Adjusters | qs to 100 |

Method of Application: The bond-repair composition is mixed directly into the oxidative hair color or bleaching formulation at a ratio of 1:10 to 1:5 (repair composition to color/bleach mixture). The formulation ensures that as oxidative reactions break disulfide bonds, the system actively repairs them in real time. The bond-repair composition provides real-time protection and repair during oxidative coloring, ensuring: Continuous restoration of disulfide bonds through HCTL and NAC-EE, Ionic bond stabilization through an ionic bonding stabilizer, pH buffering to prevent excessive protein degradation, and Hydrogen bond and antioxidant reinforcement for enhanced protection. By integrating these mechanisms, the bond-repair composition prevents excessive hair weakening, improves structural integrity, and ensures optimal color results without additional processing time.

In an alternative embodiment, the present disclosure provides an Antioxidant Protection Composition, formulated to neutralize oxidative free radicals generated during hair coloring. This composition functions alongside the bond-repair composition, providing real-time protection against protein oxidation, lipid peroxidation, and free radical-induced cuticle damage. By integrating free radical scavengers, chelating agents, stabilizers, and buffering agents, this formulation ensures that oxidative damage is minimized throughout the coloring process while maintaining compatibility with oxidative dye and bleach formulations. In one embodiment, the antioxidant protection composition includes sacrificial antioxidants that directly neutralize reactive oxygen species (ROS) before they attack keratin structures. These antioxidants include N-Acetyl Cysteine Ethyl Ester (NAC-EE), which acts as a thiol-based ROS neutralizer, reducing hydrogen peroxide-induced cysteine oxidation. NAC-EE remains inactive at neutral pH but undergoes hydrolysis at high pH (above 8.5), ensuring activation only during oxidative coloring. Another antioxidant, hydroxytyrosol, is included to scavenge superoxide radicals formed during hair coloring, preventing protein carbonylation, a major contributor to hair aging and structural weakness. Additionally, vitamin E (tocopherol) is incorporated to prevent lipid peroxidation within the cuticle, ensuring that the natural hydrophobicity of hair is preserved.

In another embodiment, the antioxidant protection composition includes chelating agents, which bind to metal ions such as copper and iron that are present in tap water, hair care products, and environmental pollutants. These metal ions accelerate free radical formation via Fenton-type reactions, leading to increased oxidative stress. The composition includes ethylenediaminetetraacetic acid (EDTA), which chelates divalent metal ions, preventing the breakdown of peroxide into damaging hydroxyl radicals. Additionally, sodium phytate, a natural plant-derived chelator, is included to bind iron and copper, preventing color fading and oxidative stress accumulation. In a further embodiment, the antioxidant protection composition contains stabilizers and buffering agents to ensure compatibility with oxidative dye and bleach formulations. These ingredients help maintain an optimal pH range and prevent destabilization of oxidative components. Sodium ascorbyl phosphate, a water-soluble vitamin C derivative, is included as a secondary antioxidant that reduces oxidative scalp irritation. Additionally, sodium gluconate functions as a pH buffer, stabilizing alkaline colorants without affecting bleach lift efficiency, ensuring consistent oxidative performance.

In an exemplary embodiment, the antioxidant protection composition comprises N-Acetyl Cysteine Ethyl Ester (NAC-EE) at a concentration of 1.0-5.0% by weight, hydroxytyrosol at 0.3-0.5%, and vitamin E at 0.3%, which collectively function to neutralize oxidative radicals and protect keratin integrity. Additionally, the formulation includes chelating agents, such as EDTA at 0.2-0.5% and sodium phytate at 0.2-0.5%, to minimize metal-induced oxidative stress. Further, sodium ascorbyl phosphate at 0.3-0.8% and sodium gluconate at 0.3-0.7% act as stabilizers, ensuring that the formulation remains effective without interfering with the coloring process. In another embodiment, the antioxidant protection composition is mixed into the oxidative hair color or bleach formulation at a ratio of 1:10 to 1:5 (antioxidant composition to color mixture). This ensures continuous neutralization of oxidative radicals while maintaining dye penetration and bleaching efficiency. The formulation remains active throughout the oxidative process, preventing structural degradation of keratin and cuticle erosion.

In a further embodiment, the antioxidant protection composition may be formulated as a standalone treatment, applied before oxidative coloring as a pre-load antioxidant system. In this approach, the antioxidant composition is applied to the hair 5-10 minutes before color application, ensuring the hair fiber is pre-saturated with antioxidants to reduce oxidative stress before it occurs. This embodiment is particularly beneficial for highly porous or chemically treated hair, where cumulative oxidative damage is a concern. The Antioxidant Protection Composition enhances the overall protective capability of the multi-phase system, ensuring real-time oxidative stress mitigation, preventing long-term protein oxidation and cuticle lipid degradation, and preserving hair integrity. This phase may be combined with the bond-repair composition or used as a standalone additive, demonstrating the flexibility of the multi-phase system while remaining within the scope of the present disclosure.

In an embodiment, the post-treatment composition is formulated to restore the cuticle, reinforce internal keratin structures, and replenish the lipid barrier after oxidative coloring. The post-treatment composition is applied after rinsing out the oxidative treatment, typically following shampooing, and functions to re-seal the hair cuticle, reduce porosity, and lock in the repaired bonds formed in the previous steps. In an embodiment, the post-treatment composition comprises an acidic pH buffer, a cuticle lipid restorer, a ceramide complex, cationic conditioning agents, and optional protein reconstructors. These components work together to normalize hair's pH, reinforce non-covalent bonds, and restore the outer lipid layer that was stripped during bleaching or dyeing.

In a specific embodiment, the post-treatment composition includes an acidic pH buffer system to neutralize residual alkalinity from the oxidative process and bring hair back to its natural acidic pH (4.0-4.5). The acidic buffer promotes cuticle contraction, allowing the scales to lie flat and form a smooth protective layer.

Suitable acidic pH buffering agents include: Citric Acid (~0.5% to 2.0%)—Lowers the pH to restore hair's natural acidic balance, Phosphoric Acid (~0.3% to 1.5%)—Prevents mineral buildup and enhances cuticle sealing, and Sodium Citrate (~0.3% to 1.0%)—Functions as a mild pH buffer while improving mineral solubility.

In an exemplary embodiment, the acidic pH buffer is included at a concentration of 1.0% to 3.0% by weight, ensuring effective cuticle realignment and porosity reduction.

In another embodiment, the post-treatment composition comprises 18-Methyl Eicosanoic Acid (18-MEA) analogs to replenish the hydrophobic F-layer of the cuticle, which is naturally present in virgin hair but is removed during oxidative coloring.

18-MEA analogs function by: Recreating hair's natural lipid barrier, reducing friction and water absorption, increasing hydrophobicity, preventing excessive swelling during future washes, and enhancing shine, smoothness, and color retention.

Suitable 18-MEA analogs include, without any limitation, 1. Behentrimonium Methosulfate (~0.5% to 1.5%)—A quaternized long-chain fatty acid that electrostatically binds to the hair surface. 2. Stearamidopropyl Dimethylamine (~0.5% to 1.5%)—Provides additional lipid-like properties and improves cuticle cohesion.

The 18-MEA analogs are included at a concentration of 0.5% to 2.5% by weight, ensuring long-lasting cuticle protection.

In another embodiment, the post-treatment composition includes ceramides and supportive lipids to repair the intercellular lipid matrix of the cuticle. Ceramides are essential for maintaining the adhesion of cuticle cells, preventing cuticle lift, and reducing moisture loss.

Suitable ceramides and lipids include: 1. Ceramide NP (~0.1% to 0.5%)—Strengthens the cuticle and prevents chipping. 2. Cholesterol (~0.3% to 1.0%)—Helps restore lipid bilayer integrity. 3. Phytosphingosine (~0.1% to 0.3%)—A natural precursor to ceramide formation, aiding in long-term cuticle repair.

In an exemplary embodiment, ceramides and lipids are included at a total concentration of 0.5% to 2.0% by weight, ensuring optimal cuticle reinforcement and moisture retention.

In another embodiment, the post-treatment composition includes cationic conditioning agents to neutralize hair's negative charge, reduce frizz, and improve manageability. These positively charged molecules bind to the negatively charged keratin sites, forming a smooth protective film on the hair surface.

Suitable cationic conditioning agents include, without any limitation, 1. Behentrimonium Chloride (~0.5% to 2.0%)—Provides conditioning and detangling properties. 2. Cetyl Trimethylammonium Chloride (~0.3% to 1.5%)—Enhances smoothness and frizz control. 3. Stearyl Amidopropyl Dimethylamine (~0.3% to 1.0%)—Acts as a mild cationic surfactant that improves lipid adherence.

The cationic conditioning agents are included at 1.0% to 3.0% by weight, ensuring immediate and long-term conditioning benefits.

In a further embodiment, the post-treatment composition includes hydrolyzed proteins to fill microscopic gaps in the cuticle and reinforce the keratin matrix.

Suitable hydrolyzed proteins include, LMW keratin or nano keratin as: 1. Hydrolyzed Keratin (~0.5% to 1.5%)—Replaces lost keratin and strengthens hair fibers. 2. Hydrolyzed Collagen (~0.3% to 1.0%)—Adds moisture retention properties. 3. Hydrolyzed Silk (~0.3% to 0.8%)—Provides an additional smooth feel.

In an exemplary embodiment, the total protein concentration is maintained at 0.5% to 2.5%, ensuring structural reinforcement without excessive stiffness.

In an exemplary embodiment, the post-treatment composition comprises:

| Ingredient | Concentration (% w/w) |
|---|---|
| Citric Acid (pH buffer) | 0.5-2.0% |
| Phosphoric Acid | 0.3-1.5% |
| Sodium Citrate | 0.3-1.0% |
| Behentrimonium Methosulfate (18-MEA analog) | 0.5-2.5% |
| Ceramide NP | 0.1-0.5% |
| Cholesterol | 0.3-1.0% |
| Phytosphingosine | 0.1-0.3% |
| Behentrimonium Chloride | 0.5-2.0% |
| Stearyl Amidopropyl Dimethylamine | 0.3-1.0% |
| Hydrolyzed Keratin | 0.5-1.5% |
| Hydrolyzed Collagen | 0.3-1.0% |
| Hydrolyzed Silk | 0.3-0.8% |
| Water & pH Adjusters | qs to 100 |

Method of Application: The post-treatment composition is applied to damp, shampooed hair and left for 3-10 minutes before rinsing. The formulation seals the cuticle, reinforces internal bonds, and restores the lipid barrier, ensuring long-term hair protection. The post-treatment composition completes the multi-phase system by sealing and reinforcing the hair structure after oxidative treatment. By integrating acidic cuticle sealing, lipid replenishment, ceramide repair, and protein reinforcement, this composition ensures lasting hair integrity, smoothness, and strength.

The present disclosure provides significant advantages in the field of hair treatment, particularly in oxidative chemical processes. The multi-phase system disclosed herein is configured to minimize structural damage to hair during oxidative treatments while maintaining the desired color results. Each phase incorporates specific compositions that function synergistically to protect and restore hair at various stages of the oxidative coloring process, ensuring enhanced structural integrity and reduced cuticle degradation. In an embodiment, the multi-phase system is particularly advantageous in professional salon applications, where it is essential to preserve hair integrity during intensive chemical treatments, such as bleaching, highlighting, balayage, and high-lift color applications. The pre-treatment composition, comprising transglutaminase, catalyzes the formation of isopeptide bonds between keratin proteins, thereby reinforcing the internal hair structure before exposure to oxidative agents. The bond-repair composition, which includes DL-homocysteine thiolactone (HCTL), functions during oxidative coloring by releasing thiols, which facilitate real-time disulfide bond reformation, thereby minimizing protein degradation. Additionally, the post-treatment composition, comprising 18-methyl eicosanoic acid (18-MEA) analogues and ceramides, restores the cuticle lipid barrier, ensuring smoothness, moisture retention, and long-term protection. These compositions collectively ensure optimal bond protection, enabling hair to maintain its structural integrity and cosmetic properties, even after multiple chemical applications.

In another embodiment, the multi-phase system may be adapted for at-home hair coloring, wherein the compositions are incorporated into a three-part or four-part system. In this embodiment, the system includes: a pre-treatment composition containing transglutaminase for keratin reinforcement before coloring; an in-dye bond-repair composition containing DL-homocysteine thiolactone (HCTL), which mitigates bond breakage during oxidative processing; a post-treatment composition comprising N-acetyl cysteine ethyl ester (NAC-EE) as an antioxidant, which functions to neutralize oxidative stress, prevent irreversible protein oxidation, and preserve hair elasticity. Optionally, an additional antioxidant protection phase that provides continuous radical scavenging during coloring to reduce cumulative oxidative damage.

The at-home system is configured to provide non-professional users with reduced hair damage, thereby enabling safer and healthier oxidative treatments compared to conventional single-step box dye formulations. Furthermore, the multi-phase system may be integrated into premium and luxury hair care products, allowing professional brands to incorporate advanced bond-building technology into exclusive salon protocols or high-end home care kits. By including transglutaminase in the pre-treatment composition, DL-homocysteine thiolactone in the bond-repair composition, and 18-MEA analogues and ceramides in the post-treatment composition, the system provides enhanced protection against oxidative damage, superior hair integrity, and optimized color longevity.

In an embodiment, the pre-treatment and post-treatment compositions are designed to integrate seamlessly with other professional hair treatments, including toning, glossing, keratin smoothing treatments, and chemical relaxers. The enzymatic crosslinking mechanism of transglutaminase in the pre-treatment composition, coupled with lipid replenishment through ceramides in the post-treatment phase, provides enhanced resistance against subsequent heat styling, chemical processing, and environmental stressors. Further, the post-treatment composition, containing ceramides and fatty acids, replenishes natural lipids, ensuring softness, smoothness, and shine, while mitigating damage associated with oxidative coloring.

In another embodiment, the multi-phase system provides cumulative protective benefits for individuals undergoing frequent oxidative treatments, such as root touch-ups, repeated highlighting, or heavy bleaching. The pre-treatment composition, comprising transglutaminase, strengthens keratin structures before each chemical service, thereby preventing progressive structural weakening. Additionally, the post-treatment sealant, containing 18-MEA analogues and ceramides, preserves color results, reduces cumulative cuticle degradation, and prevents moisture loss, ensuring healthier and stronger hair over successive treatments. In yet another embodiment, the disclosed system may be utilized for a broad range of chemical hair treatments involving alkaline oxidation, thereby ensuring stronger, healthier hair after each color application. By reducing excessive hair damage, restoring cuticle lipids, and reinforcing overall hair structure, the multi-phase system provides essential protection for both professional and at-home hair coloring applications. The incorporation of N-acetyl cysteine ethyl ester (NAC-EE) as an antioxidant, DL-homocysteine thiolactone as a thiol-based bond repair agent, and ceramides for lipid replenishment ensures that hair remains resilient, glossy, and free from oxidative stress, thereby offering a versatile and effective solution for various hair treatment applications.

In another embodiment, the multi-phase system is suitable for individuals who do not undergo oxidative coloring treatments but seek enhanced hair strength, protection, and overall scalp health. The pre-treatment composition, bond repair formulation, antioxidant protection, and post-treatment phases collectively function to fortify and protect hair from environmental stressors, mechanical damage, and heat styling effects. Additionally, these formulations contribute to scalp health, reducing the risk of irritation, sensitivity, or inflammation often associated with frequent chemical or thermal exposure. In yet another embodiment, hair treated with the multi-phase system exhibits measurable improvements in elasticity, reduced breakage, and increased tensile strength, as confirmed by clinical and laboratory testing, including microscopic imaging and hair fiber integrity assessments. These results demonstrate the efficacy of the disclosed system in providing long-term protection against chemical damage, ensuring stronger, healthier, and more resilient hair over time.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

The present disclosure is not limited to the specific embodiments, versions, or examples described herein, which are provided for purposes of illustration and to enable a person having ordinary skill in the art to make and use the invention in view of the disclosure and the knowledge generally available in the art. Modifications and variations of the described embodiments may be made without departing from the scope of the invention, which is defined solely by the appended claims.

What is claimed is:

1. A pre-treatment composition for protecting hair during oxidative coloring, the composition comprising:
a transglutaminase enzyme;
a thiol donor selected from the group consisting of N-acetyl cysteine ethyl ester (NAC-EE), DL-homocysteine thiolactone (HCTL), (R)-2-oxothiazolidine-4-carboxylic acid (OTC), and combinations thereof;
a cosmetically acceptable carrier; and
one or more of: a penetration enhancer and a conditioning agent;
wherein the composition is configured to reinforce hair structure prior to exposure to oxidative coloring agents and to activate during the oxidative coloring process to repair disulfide bonds.

2. The composition of claim 1,
wherein the transglutaminase enzyme is present in an amount ranging from about 0.3% to about 2.0% by weight of the composition;
wherein the thiol donor comprises N-acetyl cysteine ethyl ester (NAC-EE);
wherein the thiol donor comprises DL-homocysteine thiolactone (HCTL); and
wherein the thiol donor comprises (R)-2-oxothiazolidine-4-carboxylic acid (OTC).

3. The composition of claim 1,
wherein the thiol donor is present in an amount ranging from about 0.5% to about 3.0% by weight of the composition;
wherein the penetration enhancer comprises dimethyl isosorbide (DMI); and
wherein the conditioning agent is selected from the group consisting of: betaine and arginine methyl ester.

4. The composition of claim 1, wherein the pH of the composition is maintained between about 6.0 and about 7.0.

5. The composition of claim 1, further comprises at least one of: keratin peptides and protein fragments to enhance crosslinking by transglutaminase.

6. The composition of claim 1, wherein the cosmetically acceptable carrier comprises water and at least one pH-adjusting agent.

7. The composition of claim 1, wherein the composition is formulated as at least one of: a spray, lotion, serum, and cream.

8. The composition of claim 1, wherein the penetration enhancer is present in an amount ranging from about 1.0% to about 2.0% by weight of the composition.

9. The composition of claim 1, wherein the composition further comprises a buffering agent to maintain pH stability during storage.

10. The composition of claim 1, wherein the thiol donor is activated under alkaline oxidative conditions having a pH greater than 8.5.

11. The composition of claim 1, wherein the composition is configured for application to hair for a period ranging from about two hours to overnight before oxidative coloring.

12. The composition of claim 1, wherein the composition forms internal isopeptide bonds within keratin fibers prior to exposure to oxidative coloring agents.

13. A method for protecting hair during oxidative coloring, the method comprising:
applying a pre-treatment composition to hair, wherein the pre-treatment composition comprising a transglutaminase enzyme, a thiol donor selected from the group consisting of N-acetyl cysteine ethyl ester (NAC-EE), DL-homocysteine thiolactone (HCTL), (R)-2-oxothiazolidine-4-carboxylic acid (OTC), and combinations thereof, and a cosmetically acceptable carrier; and
subsequently subjecting the hair to an oxidative coloring treatment without removing the pre-treatment composition;
wherein the pre-treatment composition reinforces the hair structure prior to exposure to oxidative coloring agents and activates during the oxidative coloring process to repair disulfide bonds.

14. The method of claim 13, wherein the pre-treatment composition is applied to the hair for a duration ranging from about two hours to overnight prior to oxidative coloring.

15. The method of claim 14, wherein the pre-treatment composition is not rinsed off prior to performing the oxidative coloring treatment.

16. The method of claim 14, wherein the oxidative coloring treatment comprises at least one of: bleaching, highlighting, and dyeing using hydrogen peroxide-based compositions.

17. The method of claim 14, wherein the thiol donor comprises N-acetyl cysteine ethyl ester (NAC-EE).

18. The method of claim 17, wherein activation of the thiol donor occurs under alkaline oxidative conditions having a pH greater than about 8.5.

19. The method of claim 13, wherein the pre-treatment composition is formulated as at least one of: a lotion, spray, serum, and cream.

20. The method of claim 13, wherein the pre-treatment composition reduces hair breakage during oxidative coloring by maintaining disulfide bond integrity.
